(19) United States
(12) Patent Application Publication
(10) Pub. No.: US 2024/0094195 A1
(43) Pub. Date: Mar. 21, 2024

(54) METHOD FOR ASSESSING RESPIRATORY VIRAL INFECTIONS

(71) Applicants: Centre for Immunology & Infection Limited, Hong Kong (HK); The University of Hong Kong, Hong Kong (HK)

(72) Inventors: Chi Wai Michael CHAN, Hong Kong (HK); Pui Yan HUI, Hong Kong (HK); John Malcolm NICHOLLS, Hong Kong (HK); Hiu Ha CHING, Hong Kong (HK); Hau Yin Kevin FUNG, Hong Kong (HK)

(21) Appl. No.: 18/466,854

(22) Filed: Sep. 14, 2023

Related U.S. Application Data
(60) Provisional application No. 63/406,742, filed on Sep. 15, 2022.

(51) Int. Cl. G01N 33/52; C12N 5/071; G01N 1/28

(52) U.S. Cl. CPC G01N 33/52; C12N 5/0688; G01N 1/286; C12N 2500/32; G01N 2001/2873; G01N 2474/20; G01N 2800/26

(57) ABSTRACT

The present invention relates to a method for assessing respiratory viral infections, the method includes preparing one or more precision-cut human bronchial tissues; exposing a cilia-rich epithelium within the precision-cut human bronchial tissues to establish an ex vivo model of human bronchus for viral infections; assessing infectivity, tropism, and pathogenesis of one or more respiratory viruses in the ex vivo model; and providing a semi-quantitative and normalized approach to supplement data from the ex vivo model of human bronchus for pandemic risk assessment of the one or more respiratory viruses. The present invention involves micro-dissection of bronchial tissues to expose the cilia-rich epithelium for ex vivo virus infection, along with a semi-quantitative approach for analyzing virus tropism and replication competence.

### CROSS-REFERENCE TO RELATED APPLICATIONS

The present application claims priority from U.S. provisional patent application Ser. No. 63/406,742 filed Sep. 15, 2022, and the disclosure of which is incorporated herein by reference in its entirety.

### FIELD OF THE INVENTION

The present invention generally relates a micro-dissection of bronchial tissues to expose the cilia-rich epithelium for the ex vivo virus infection; and it further relates to a semi-quantitative approach for analyzing virus tropism and replication competence.

### BACKGROUND OF THE INVENTION

Respiratory infections are a prominent cause of global mortality. Much of the research into the underlying disease mechanisms relies on cell culture, organoid, or surrogate animal models. While these approaches offer valuable insights, they are not without limitations. For example, traditional cell culture models poorly reflect the cellular composition, matrix complexity, and three-dimensional architecture of the human lung. Animal models are affected by species differences, this is a factor of particular significance when studying zoonotic lung diseases. The utilization of ex vivo human lung tissue could potentially address certain limitations and complement traditional models.

In recent decades, the ex vivo tissue models take a crucial role in the investigation of emerging viral zoonoses, including influenza A virus and coronaviruses. In numerous tissues, the apical surface of cells (epithelium) is oriented towards the external environment or lumen, making it a primary target site for respiratory viral infection. Hence, when employing the human airway as an ex vivo model to investigate the tropism and pathogenesis of emerging respiratory viruses, the tissue preparation and dissection process becomes crucial to expose the epithelium for virus attachment, entry, and infection.

Pandemics emerge at unpredictable intervals and can spread worldwide within weeks due to high infection attack rates, potentially leading to significant morbidity and mortality¹. Vaccines will not be available in time to mitigate the first wave of pandemic. Therefore, active surveillance programs have been carried out worldwide to identify and assess the risk of animal viruses circulating in wild birds, poultry, swine and other animals, so that seed viruses for candidate vaccine and sometimes actual vaccines can be prepared pre-emptively. The 2009 pandemic virus emerged as a result of the reassortment between swine North American triple reassortment H1N1/H1N2 viruses (which had caused zoonotic diseases in North America) and swine viruses of the Eurasian-avian lineage². The failure to identify these swine North American viruses as potential pandemic threats is not due to a lack of surveillance, but rather a gap in risk assessment. Since then, algorithms have been developed for systematic risk assessment of animal influenza viruses for pandemic threat to facilitate the selection of animal viruses as vaccine candidates.

CDC Influenza Risk Assessment Tool (IRAT) and WHO Tool for Influenza Pandemic Risk Assessment (TIPRA) are used to evaluate viruses for pandemic threat in relations to emergence risk and public health impact or disease severity^{3,4}. These assessments rely on virus properties, population attributes, and virus ecology. The individual parameters contributing to these algorithms require further refinement.

At present, this parameter is indirectly evaluated by studying the binding of virus to sialic acids that are α2-6-linked to galactose (Gal), as these are believed to be the type of receptors present in the human upper airways⁵. However, there is a wide diversity of mono-antennary, bi-antennary or poly-antennary glycans, and it is still unclear which of these are present in the human upper airways⁶.

Certain glycans found in the human upper airways are not currently available in synthetic form and are not included in the glycan arrays used to assess receptor binding of influenza viruses. As a result, an alternative and more direct approach has been developed, involving the use of ex vivo cultures of human upper airways to study the tropism and replication competence of influenza viruses⁷⁻⁹.

Virus binding to bronchial tissues¹⁰ or infection of ex vivo human bronchus⁷⁻⁹ shows a strong correlation with viruses that demonstrate airborne transmission in ferrets, which is another reliable indicator of transmissibility in humans. Due to the potential for donor variability, comparing viral replication competence across separate experiments of virus-infected ex vivo cultures in a consistent manner has proven challenging.

Accordingly, there is a need in the art to provide a new precision-cut human bronchial tissue as ex vivo infection model for the risk assessment of respiratory viral infections. The present invention addresses this need.

### SUMMARY OF THE INVENTION

Accordingly, the present invention aims to address the challenges associated with ex vivo cultures of human upper airways.

The present invention offers a method for quantitatively analyzing the viral replication competence of a selection of influenza viruses within the human respiratory tract. This analysis is conducted by comparing their performance with two reference virus strains that are recognized for efficient human transmission: the 2009 H1N1 pandemic virus and highly pathogenic avian influenza (HPAI) H5N1 virus.

In a first aspect, the present invention provides a method for assessing respiratory viral infections, the method includes preparing one or more precision-cut human bronchial tissues; exposing a cilia-rich epithelium within the precision-cut human bronchial tissues to establish an ex vivo model of human bronchus for viral infections; assessing infectivity, tropism, and pathogenesis of one or more respiratory viruses in the ex vivo model; and providing a semi-quantitative and normalized approach to supplement data from the ex vivo model of human bronchus for pandemic risk assessment of the one or more respiratory viruses.

In accordance with one embodiment, the one or more precision-cut human bronchial tissue is prepared according to the following steps: collecting one or more bronchial tissues from at least one patient with lung carcinoma who underwent surgical resection of lung tissue; washing the one or more bronchial tissues with PBS twice to remove blood contaminants; transferring the one or more bronchial tissues onto at least one petri dish; cutting away unwanted tissues attached to the bronchial tissues; washing the bronchial tissues with PBS for 1-2 times to remove surface-attached mucus; performing one lengthwise cut to open bronchial tubes to expose a luminal surface of an airway epithelium, wherein the airway epithelium is faced upwards; cutting opened bronchial tubes having exposed luminal surface of the airway epithelium into squares, and obtaining the one or more precision-cut human bronchial tissue.

In accordance with one embodiment, the one lengthwise cut is performed using a sterile surgical fine scissor and forceps.

In accordance with one embodiment, the method further includes placing the one or more precision-cut human bronchial tissue with the airway epithelium facing upwards onto one or more sterile surgical pathology sponges to establish the ex vivo model with an air-liquid interface condition in a 24-well culture plate.

In accordance with one embodiment, a culture medium is added to each well and the culture plate is incubated at 37° C. for subsequent viral infection experiments.

In accordance with one embodiment, the culture medium includes F-12K nutrient mixture with L-glutamine and at least one antibiotic.

In accordance with one embodiment, the unwanted tissues include soft tissues and elastic blood vessels.

In accordance with one embodiment, the one or more respiratory viruses include influenza A viruses, influenza B viruses, coronaviruses, or any combinations of influenza viruses thereof.

In accordance with one embodiment, the virus infection is performed by subjecting the cilia-rich epithelium within the precision-cut human bronchial tissues to 10⁶ TCID₅₀/mL infectious dose of the one or more respiratory viruses and incubated at 37° C. and 5% CO₂ incubator.

In accordance with one embodiment, the step of assessing infectivity, tropism, and pathogenesis of one or more respiratory viruses in the ex vivo model further includes collecting tissue supernatants at 1 hour, 24 hours and 48 hours post-infection for measuring viral replication.

In accordance with one embodiment, the step of assessing infectivity, tropism, and pathogenesis of one or more respiratory viruses in the ex vivo model further includes fixing the precision-cut human bronchial tissues with 10% formalin at 24 hours post-infection or 48 hours post-infection in preparation for immunohistochemistry staining.

In accordance with one embodiment, the step of providing a semi-quantitative and normalized approach includes utilizing a post-staining or scoring analysis to supplement data from the ex vivo cultures of human bronchus.

### BRIEF DESCRIPTION OF THE DRAWINGS

Embodiments of the present invention are described in more detail hereinafter with reference to the drawings, in which:

FIG. 1 depicts the preparation of precision-cut human bronchial tissue as an ex vivo infection model;

FIGS. 2A and 2B depicts replication competency of influenza viruses and coronaviruses in ex vivo cultures of human bronchus. The relative area under curve of human bronchus replication kinetic was compared by one-way ANOVA with Bonferroni's post-test. *P<0.05, **P<0.01, ***P<0.001 and ns=not significant; and

FIG. 3 shows the tissue tropism of influenza viruses and coronaviruses within ex vivo cultures of human bronchi.

### DETAILED DESCRIPTION

The present invention will be described in detail through the following embodiments with appending drawings. It should be understood that the specific embodiments are provided for an illustrative purpose only, and should not be interpreted in a limiting manner. Those skilled in the art will appreciate that the invention described herein is susceptible to variations and modifications other than those specifically described.

The invention includes all such variation and modifications. The invention also includes all of the steps and features referred to or indicated in the specification, individually or collectively, and any and all combinations or any two or more of the steps or features. Other aspects and advantages of the invention will be apparent to those skilled in the art from a review of the ensuing description.

The emergence of animal influenza viruses circulating in both poultry and human populations poses a significant public threat. In numerous tissues, the apical surface of the epithelium serves as a primary target site for respiratory viral infection. One key parameter with heavy weighting for assessing the risk of a virus acquiring transmissibility in humans is the potential to infect and replicate in the human upper airways. Currently, this is largely assessed indirectly by virus binding to receptors found on the epithelial cells of the upper airways. However, it is difficult to identify the region of airway epithelium. The airway epithelium could sustain damage during the dissection process, and there is a risk of incorrect tissue orientation in the air-liquid interface (ALI) system where the epithelium may not be facing the air. Currently, the risk assessment tools that link the gap between surveillance and their risk in human transmission and disease severity are lacking.

When utilizing the human airway as an ex vivo model to study the tropism and pathogenesis of emerging respiratory viruses, the tissue preparation and dissection process becomes crucial to ensure the exposure of the epithelium for virus attachment, entry, and infection. Accordingly, the present invention provides a method for assessing respiratory viral infections, the method includes preparing one or more precision-cut human bronchial tissues; exposing a cilia-rich epithelium within the precision-cut human bronchial tissues to establish an ex vivo model of human bronchus for viral infections; assessing infectivity, tropism, and pathogenesis of one or more respiratory viruses in the ex vivo model; and providing a semi-quantitative and normalized approach to supplement data from the ex vivo model of human bronchus for pandemic risk assessment of the one or more respiratory viruses.

In one of the embodiments, the one or more precision-cut human bronchial tissue is/are prepared according to the following steps: collecting one or more bronchial tissues from at least one patient with lung carcinoma who underwent surgical resection of lung tissue; washing the one or more bronchial tissues with PBS twice to remove blood contaminants; transferring the one or more bronchial tissues onto at least one petri dish; cutting away unwanted tissues attached to the bronchial tissues; washing the bronchial tissues with PBS for 1-2 times to remove surface-attached mucus; performing one lengthwise cut to open bronchial tubes to expose a luminal surface of an airway epithelium, wherein the airway epithelium is faced upwards; cutting opened bronchial tubes having exposed luminal surface of the airway epithelium into squares (˜5 mm in length), and obtaining the one or more precision-cut human bronchial tissue.

In one of the embodiments, the precision-cut human bronchial tissues have an average length of 5 mm.

In one of the embodiments, the respiratory viral infections may include, but are not limited to, influenza and coronaviruses.

In one of the embodiments, the viruses may be influenza A viruses. Influenza A viruses are classified based on different combinations of hemagglutinin (H) and neuraminidase (N) surface proteins. The common subtypes of influenza A may be H1N1, H3N2, H2N2, and H5N1.

In one of the embodiments, the viruses may be influenza B viruses. Influenza B viruses are a type of virus that causes seasonal flu and often co-circulate with influenza A viruses. Influenza B viruses are typically classified into two main lineages, namely the Yamagata lineage and the Victoria lineage. Each lineage consists of different subtypes and strains: (1) Yamagata lineage: This is a major branch of influenza B viruses that commonly cause seasonal flu. It includes a range of subtypes and strains such as B/Yamagata/16/88, and more. (2) Victoria lineage: Similarly, the Victoria lineage is another major branch of influenza B viruses responsible for seasonal flu. Within this lineage, there are various subtypes and strains like B/Victoria/2/87, and others.

In one of the embodiments, the viruses may be coronaviruses. Coronaviruses are classified into different species and genera and are characterized by their specific appearance. The commonly known types of coronaviruses: (1) Human Coronaviruses: These include coronaviruses that cause mild respiratory infections, such as 229E, NL63, OC43, and HKU1. (2) Severe Acute Respiratory Syndrome Coronavirus (SARS-CoV). (3) Middle East Respiratory Syndrome Coronavirus (MERS-CoV). (4) Severe Acute Respiratory Syndrome Coronavirus 2 (SARS-CoV-2).

The following examples illustrate the present invention and are not intended to limit the same.

### EXAMPLE

### Example 1—Materials and Methods

Influenza Viruses and Coronaviruses

Influenza viruses and coronaviruses, as well as their origins of virus isolation, were listed in Table 1. Wild bird fecal samples were collected during routine surveillance at the Hong Kong Mai Po Nature Reserve. All influenza viruses were passaged in Madin-Darby Canine Kidney (MDCK) cells, while coronaviruses were passaged in Vero E6 or MRC-5 cells. Viral titers were determined by median tissue culture infectious dose (TCID₅₀)⁸. All experiments were performed inside a biosafety level-3 facility.

Viral Titration Using the TCID₅₀ Assay

First, confluent 96-well tissue culture plates of MDCK, Vero E6, or MRC-5 cells were prepared one day in advance. The cells were washed once with PBS and then provided with serum-free Minimum Essential Media (MEM) for MDCK cells, Dulbecco's Modified Eagle's Medium (DMEM) for Vero E6 cells, and 2% MEM for MRC-5 cells. These media were supplemented with 100 units/ml penicillin, 100 μg/ml streptomycin, and 2 μg/ml of TPCK (tosylsulfonyl phenylalanylchloromethyl ketone)-treated trypsin.

Serial dilutions of the virus supernatant, ranging from 0.5 log to 7 log, were carried out prior to adding them to the cell plates. Then, the plates were placed in a 37° C. and 5% CO₂ humidified incubator, and daily observations were made for the development of cytopathic effects. The endpoint of viral dilution resulting in cytopathic effects (CPE) in 50% of inoculated wells was determined using the Karber method¹⁴.

Statistical Analysis

Ex vivo infections of multiple viruses were compared using one-way ANOVA with Bonferroni post-tests in Prism software.

### Example 2—Preparation of Precision-Cut Human Bronchial Tissues as Ex Vivo Infection Model

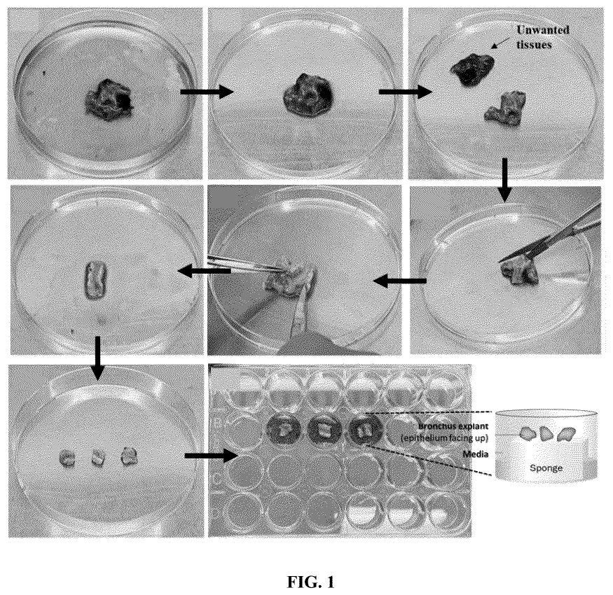

FIG. 1 showed the preparation of precision-cut human bronchial tissues. Bronchial tissues were obtained from patients with lung carcinoma who underwent surgical resection of lung tissue. Tissue fragments of normal, non-malignant tissue that exceeded the clinical diagnosis requirements were utilized.

First, the bronchial tissues were washed with PBS twice in order to remove blood contaminants. The tissue was then transferred onto a petri dish, and sterile surgical scissors were used to cut away the unwanted tissues (soft tissues and elastic blood vessels) attached to the bronchi. After that, the bronchi were washed with PBS for 1-2 times to remove as much surface-attached mucus as possible. Sterile surgical fine scissors and forceps were use to perform one lengthwise cut to open the bronchial tube to expose the luminal surface (of airway epithelium). The bronchus was then opened so that it lay flat with the epithelium facing upwards. Forceps and a scalpel were used to cut them into square explants approximately 5 mm in length. Usually, the two ends of the tube were not used due to harsh handling (where the epithelium may be damaged). During the cutting process, care should be taken to avoid stretching, pressing, and damaging the epithelial layer. Finally, the bronchial explants (epithelium facing upwards) were placed on sterile surgical pathology sponges to establish an air-liquid interface (ALI) condition in a 24-well culture plate. Culture medium (F-12K nutrient mixture (Gibco) with L-glutamine, and antibiotics such as 100 U/ml penicillin and 100 g/ml streptomycin) was added to each well and the plates was incubated at 37° C. for subsequent viral infection experiments.

### Example 3—Infection of Ex Vivo Cultures of Human Bronchus

Tissue maintenance, infection protocol, and analysis were performed as previously described^{7-9, 13}. Following the sectioning of human bronchi into thin pieces, they were subjected to 10⁶ TCID₅₀/mL infectious dose of different viruses, and incubated at 37° C. and 5% CO₂ incubator. Tissues were rinsed off unbound viruses with PBS after 1 hour incubation. Bronchus tissues were then placed into a 24-well plate containing F-12K nutrient mixture, supplemented with 100 U/ml penicillin and 100 g/ml streptomycin. After that, the bronchus were placed on top of a surgical sponge to establish an air-liquid interface (ALI). The infected tissues were placed back to the 37° C. incubator. Tissue supernatants were collected at 1-, 24- and 48-hour post-infection (hpi) for viral replication, and the tissues were fixed with 10% formalin at 24 hours post-infection (hpi) for influenza A and at 48 hpi for influenza B and coronaviruses, in preparation for immunohistochemistry. The infectivity of both ciliated and non-ciliated epithelial cells was evaluated by a clinical pathologist.

### Example 4—Area Under Curve (AUC) Analysis of Viral Replication Competence in Ex Vivo Culture

Table 1 showed a list of influenza viruses and coronaviruses evaluated for tissue tropism in ex vivo explant infection. The subtypes, lineages, and strains of influenza A, B, and coronaviruses, along with their respective abbreviations were listed. The viruses were isolated from avian surveillance conducted at the Hong Kong Mai Po Natural Reserve. Seasonal influenza A H1N1, H3N2, influenza B, MERS-CoV and SARS-CoVs had productive bronchus viral replication and tissue infection, but minimal for wild bird surveillance isolates H5N3 and H7N1 when referenced to pandemic H1N1 and HPAI H5N1. The findings revealed an important association between viral tropism and human transmission in ex vivo explants.

TABLE 1
Influenza viruses and coronaviruses evaluated for tissue tropism in ex vivo explant infection

| Virus Strain | Abbreviation | Subtype | Virus isolation origin |
|---|---|---|---|
| Influenza A | | | |
| A/Hong Kong/54/1998 | H1N1 (54/98) | H1N1 | Human |
| A/Oklahoma/447/2008 | H1N1 (447/08) | H1N1 | Human |
| A/Hong Kong/415742/2009 | H1N1pdm (415742/09) | H1N1pdm | Human |
| A/Hong Kong/1174/1999 | H3N2 (1174/99) | H3N2 | Human |
| A/Oklahoma/1992/2005 | H3N2 (1992/05) | H3N2 | Human |
| A/Hong Kong/483/1997 | H5N1 (483/97) | H5N1 | Human |
| A/Vietnam/1203/2004 | H5N1 (1203/04) | H5N1 | Human |
| A/Shenzhen/1/2012 | H5N1 (SZ1/12) | H5N1 | Human |
| A/Hong Kong/MPQ1017/2015* | H5N3 (MPQ1017/15) | H5N3 | Wild bird/Avian |
| A/Guangzhou/39715/2014 | H5N6 (39715/14) | H5N6 | Human |
| A/Oriental magpie robin/Hong Kong/ | H5N6 (6154/15) | H5N6 | Wild bird/Avian |
| 6154/2015 | | | |
| A/Northern pintail/Hong Kong/ | H5N8 (MP5583/04) | H5N8 | Duck/Avian |
| MP5883/2004 | | | |
| A/Hong Kong/MPQ1219/2015* | H7N1 (MPQ1219/15) | H1N1 | Wild bird/Avian |
| A/Shanghai/1/2013 | H7N9 (Sh1/13) | H7N9 | Human |
| A/Shanghai/2/2013 | H7N9 (Sh2/13) | H7N9 | Human |
| A/Anhul/1/2013 | H7N9 (AH1/13) | H7N9 | Human |
| A/Qingyuan/GIRD1/2017 | H7N9 (QY/17) | H7N9 | Human |
| A/Quail/Hong Kong/G1/1997 | H9N2 (G1/97) | H9N2 | Quail/Avian |
| A/Duck/Hong Kong/Y280/1997 | H9N2 (Y280/97) | H9N2 | Duck/Avian |
| Influenza B | | Lineage | |
| 8/Hong Kong/407373/2011 | B (407373/11) | Victoria | Human |
| B/Hong Kong/448799/2012 | B (448799/12) | Yamagata | Human |
| Coronavirus | | Strain | |
| HCoV-EMC/2012 | MERS-CoV | MERS-CoV | Human |
| SARS-CoV | SARS-CoV | HK39849 | Human |

Viral replication competence in ex vivo cultures of human bronchi was presented as the area under the curve (AUC). AUC is defined by the area between TCID₅₀ detection limit and the replication kinetic curve at 24 and 48 hpi (n≥3).

The viral titer in the culture supernatant of infected tissues at 1, 24 and 48 hpi obtained from TCID₅₀ assay was entered into the GraphPad Prism software. Total virus release from infected tissues was calculated from the trapezoid area under virus replication kinetic curves to the detection limit of TCID₅₀ (10^{1.5}) between 24 and 48 hpi¹⁵. Infectious viral titers at 1 hpi may reflect the initial inoculum and were consequently not included in the calculation of the AUC.

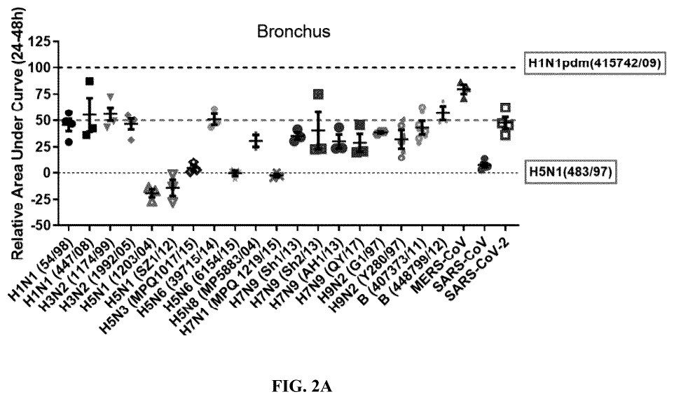

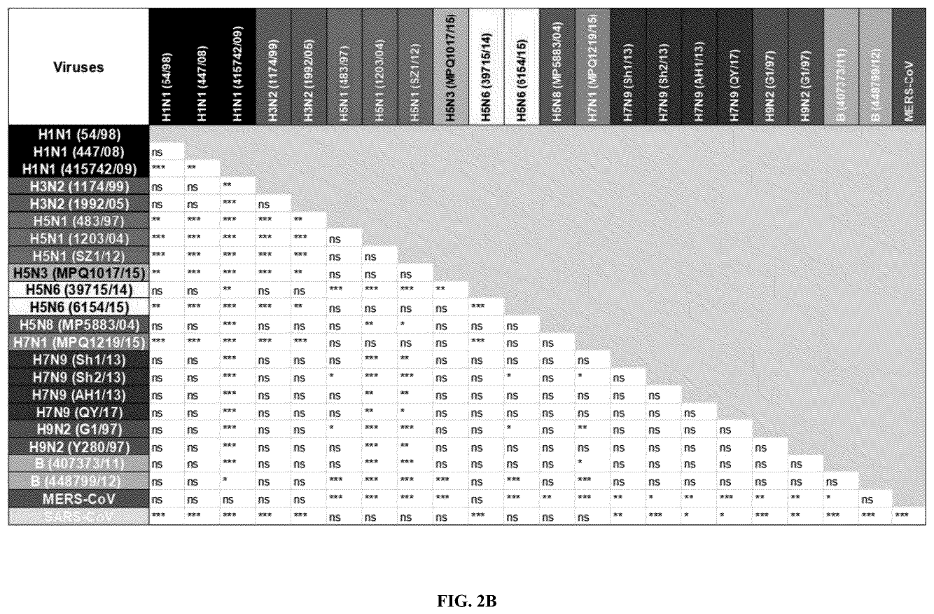

Referring to FIGS. 2A-2B, the AUC of the reference strain pandemic H1N1 (A/Hong Kong/415742/2009) was established as 100, while HPAI H5N1 (A/Hong Kong/483/1997) was set as 0 for each replicate experiment. The middle dotted line represents 50% relative AUC. The calculated AUC of each test virus was normalized to these reference strains using the following calculation:

(AUC_{VIRUS}−AUC_{H5N1})/AUC_{H1N1},

The relative AUC index was depicted as a dot plot with mean±SEM using GraphPad Prism. Each virus was tested using at least three independent donors of human bronchial tissues.

Seasonal influenza A, B and MERS-CoV had substantial replication on ex vivo human bronchus cultures with relative mean area under curve (AUC) ≥41.1 (minimal AUC index). Since the 2009 H1N1 pandemic is the most recently emerged pandemic virus that has shown sustained human-to-human transmission whereas HPAI H5N1 viruses have not become transmissible in humans in spite of repeated zoonotic infections for over two decades, these two viruses were used as the “positive” and “negative” control reference strains for AUC normalization of different experiments⁸⁻¹¹. Seasonal human influenza H1N1 (54/98, 447/08), H3N2 (1174/99, 1992/05), H5N6 (39715/14), influenza B viruses (407373/11, 448799/12), MERS-CoV (EMC) and SARS-CoV-2 had around 50% relative AUC with the lowest AUC index of 42.97. In contrast, HPAI H5N1 (1203/04, SZ1/12) and LPAI surveillance isolates H5N3 (MPQ1017/15), H7N1 (MPQ1219/15), H5N6 (6154/15) and SARS-CoV had relative AUC comparable to the control HPAI H5N1 (483/97) virus. Human isolate HPAI H5N6 (39715/14) had markedly higher bronchus replication than avian HPAI H5N6 (6154/15) virus, this was similar to the previous study¹². Interestingly, H7N9 (Sh1/13, Sh2/13, AH1/13, QY/17) and H9N2 (G1/97, Y280/97) had around 40% relative AUC comparable with some seasonal influenza viruses. MERS-CoV replicated better in human bronchus than SARS-CoV.

### Example 5—Tissue Tropism Evaluation

In this example, immunohistochemical staining was performed. The human bronchi tissues were fixed with 10% formalin overnight at 4° C. and the fixed tissues were embedded in paraffin blocks at 24 hpi (for influenza A) and 48 hpi (for influenza B and coronaviruses). The 4 μm-thick sliced sections were subjected to incubation with 0.05 mg/ml Pronase or microwaved for 15 minutes for antigen retrieval. Endogenous peroxidase activity was stopped by quenching the tissue sections with 3% H₂O₂ for 20 minutes. Subsequently, the slides were blocked with 10% normal horse serum at room temperature (RT) and incubated with primary antibodies including HB65, Influenza A Nucleoprotein (NP) antibody and SARS-CoV NP antibody, for a duration of 90 minutes at RT. This was followed by incubation with peroxidase (HRP)-conjugated anti-rabbit antibody (Vector Laboratory). The sections were developed using NovaRED Substrate Kit (Vector Laboratory). The cell nuclei were counterstained with Mayer's Hematoxylin.

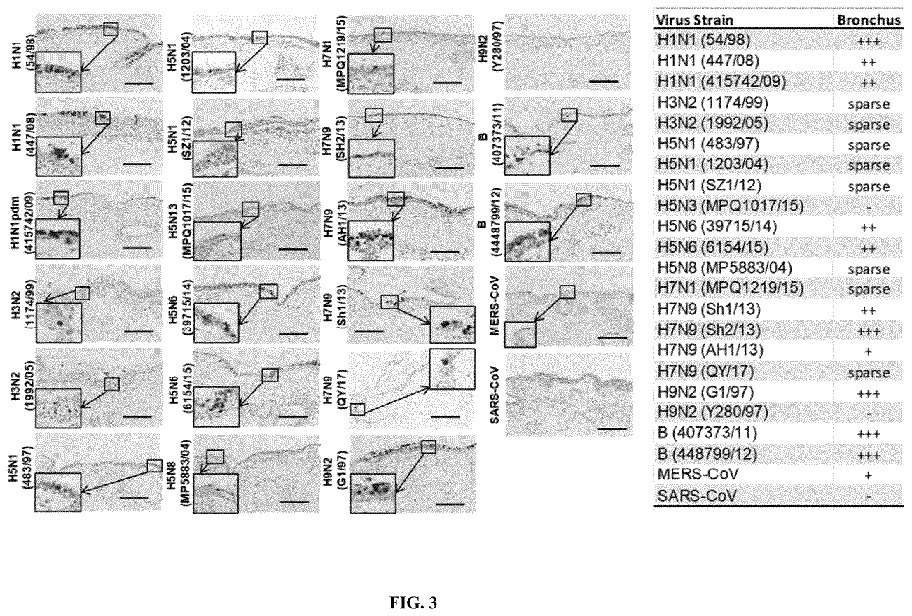

FIG. 3 showed that sections were immunohistochemically-stained for nucleoprotein with specific antibodies against respective viral nucleoprotein. The magnification is 200×, and the scale bar represents 100 μm. The pattern of antigen-positive cells (including ciliated and non-ciliated epithelial cells) of human bronchus in the IHC staining suggested different levels of infectivity:
(1) Antigen-positive cells (e.g., seasonal H1N1, H1N1 pandemic, H7N9 (Sh2/13), H9N2 (G1/97) and influenza B viruses) accounting for ≥61% of total epithelial cells were classified as high infection;
(2) Antigen-positive cells (e.g., H5N6, H7N9 (Sh1/13) and MERS-CoV) accounting for 31-60% were considered moderate; and
(3) Antigen-positive cells (e.g., HPAI H5N1, H5N3, H5N8, H7N1, H7N9 (AH1/13, QY/17), H9N2 (Y280/97) and SARS-CoV) accounting for 1-30% were categorized as minimal infection.

The table on the right side displayed the immunohistochemical scoring of infected human bronchus. The extent of viral infectivity was indicated by the following criteria:
(sparse)≤10%,
(+) 11-40%,
(++) 41-70%, and
(+++)≥71% of the total epithelial cells;
(−) denoted the absence of positively stained epithelial cells.

Additionally, H3N2 (1174/99, 1992/05) viruses had shown a low number of infected bronchial epithelial cells, even though they had demonstrated moderate viral replication. Generally, there were no notable differences in infectivity by morphological analysis between ciliated and non-ciliated epithelial cells among the viruses.

In summary, the precision-cut airway tissue of the present invention serve as an ex vivo organotypic model, which retains remarkable cellular complexity and the architecture, and thus providing a platform to investigate respiratory pathogens. The present invention employs a semi-quantitative approach to analyze virus tropism and replication competence, while also conducting a risk assessment of the adaptation of influenza and coronaviruses to transmit between humans using an ex vivo model.

### Definitions

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The term “at. %” stands for “atomic percent,” which is a unit used to describe the relative content of chemical components. In chemistry and materials science, atomic percent represents the percentage of a certain element's atoms in a compound or mixture, calculated based on the number of atoms of that element. It can be used to describe the relative proportions of different elements in solid materials, alloys, solutions, and more. Atomic percent provides a more precise way to indicate the content of elements, especially in cases involving complex chemical compositions and alloy formulations, as it accurately reflects the relative contributions of elements.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

It will be appreciated by those skilled in the art, in view of these teachings, that alternative embodiments may be implemented without undue experimentation or deviation from the spirit or scope of the invention, as set forth in the appended claims. This invention is to be limited only by the following claims, which include all such embodiments and modifications when viewed in conjunction with the above specification and accompanying drawings.

### INDUSTRIAL APPLICABILITY

The present method finds industrial applicability in the field of virology, respiratory medicine, and epidemiology, as it addresses a crucial need for effective evaluation and risk assessment of respiratory viral infections. By utilizing precision-cut human bronchial tissues and establishing an ex vivo model, the present method allows for controlled and reproducible experimentation, facilitating a comprehensive understanding of virus behavior in a relevant physiological context. This, in turn, can contribute to the development of novel therapeutic interventions, vaccines, and strategies to combat respiratory viruses.

References: The disclosures of the following references are incorporated by Reference
1. Webster, R. G., et al., Influenza pandemics: History and lessons learned. 2nd ed. Textbook of Influenza. Vol. Chapter 2. 2013, New York: New York: John Wiley & Sons, Incorporated. 20-33.
2. Smith, G. J., et al., Origins and evolutionary genomics of the 2009 swine-origin H1N1 influenza A epidemic. Nature, 2009. 459(7250): p. 1122-5.
3. CDC. Influenza Risk Assessment Tool (IRAT). 2016 9 Oct. 2019 27 Nov. 2019]; Available from: www.cdc.gov/flu/pandemic-resources/national-strategy/risk-assessment.htm.
4. WHO. Tool for Influenza Pandemic Risk Assessment (TIPRA). 2016 May 2016 Apr. 11 2018]; Available from: http://apps.who.int/iris/bitstream/handle/10665/250130/WHO-OHE-PED-GIP-2016.2-eng.pdf;jsessionid=9B3AC4999DA51F50416C26E2E760B304?sequence=1.
5. Gagneux, P., et al., Human-specific regulation of alpha 2-6-linked sialic acids. The Journal of biological chemistry, 2003. 278(48): p. 48245-50.
6. Walther, T., et al., Glycomic Analysis of Human Respiratory Tract Tissues and Correlation with Influenza Virus Infection (Respiratory Tract Glycans and Influenza). PLoS Pathog, 2013. 9(3): p. e1003223.
7. Chan, M. C., et al., Tropism and innate host responses of a novel avian influenza A H7N9 virus: an analysis of ex-vivo and in-vitro cultures of the human respiratory tract. Lancet Respir Med, 2013. 1(7): p. 534-42.
8. Chan, M. C., et al., Tropism and innate host responses of the 2009 pandemic H1N1 influenza virus in ex vivo and in vitro cultures of human conjunctiva and respiratory tract. Am J Pathol, 2010. 176(4): p. 1828-40.
9. Chan, R. W., et al., Use of ex vivo and in vitro cultures of the human respiratory tract to study the tropism and host responses of highly pathogenic avian influenza A (H5N1) and other influenza viruses. Virus Res, 2013. 178(1): p. 133-45.
10. van Riel, D., et al., Human and avian influenza viruses target different cells in the lower respiratory tract of humans and other mammals. Am J Pathol, 2007. 171(4): p. 1215-1223.
11. Farrar, J., A. Han, and F. Hayden, Avian Influenza A (H5N1) Infection in Humans. The New England Journal of Medicine, 2005. 353(13): p. 1374-85.
12. Hui, K. P., et al., Tropism and innate host responses of influenza A/H5N6 virus: an analysis of ex vivo and in vitro cultures of the human respiratory tract. Eur Respir J, 2017. 8(49): p. 3.
13. Chan, R. W., et al., Tropism and replication of Middle East respiratory syndrome coronavirus from dromedary camels in the human respiratory tract: an in-vitro and ex-vivo study. Lancet Respir Med, 2014. 2(10): p. 813-22.
14. Karber, G., Beitrag zur kollektiven Behandlung pharmakologischer Reihenversuche. Naunyn-Schmiedebergs Archiv für experimentelle Pathologie und Pharmakologie, 1931. 162(4): p. 480-483.
15. Gonzalez-Parra, G., T. Rodriguez, and H. M. Dobrovolny, A comparison of methods for extracting influenza viral titer characteristics. J Virol Methods, 2016. 231: p. 14-24.

What is claimed is:

1. A method for assessing respiratory viral infections, the method comprises:
preparing one or more precision-cut human bronchial tissues;
exposing a cilia-rich epithelium within the precision-cut human bronchial tissues to establish an ex vivo model of human bronchus for viral infections; and
assessing infectivity, tropism, and pathogenesis of one or more respiratory viruses in the ex vivo model; and
providing a semi-quantitative and normalized approach to supplement data from the ex vivo model of human bronchus for pandemic risk assessment of the one or more respiratory viruses.

2. The method of claim 1, wherein the one or more precision-cut human bronchial tissue is prepared according to the following steps:
collecting one or more bronchial tissues from at least one patient with lung carcinoma who underwent surgical resection of lung tissue;
washing the one or more bronchial tissues with PBS twice to remove blood contaminants;
transferring the one or more bronchial tissues onto at least one petri dish;
cutting away unwanted tissues attached to the bronchial tissues;
washing the bronchial tissues with PBS for 1-2 times to remove surface-attached mucus;
performing one lengthwise cut to open bronchial tubes to expose a luminal surface of an airway epithelium, wherein the airway epithelium is faced upwards;
cutting opened bronchial tubes having exposed luminal surface of the airway epithelium into squares, and obtaining the one or more precision-cut human bronchial tissue.

3. The method of claim 2, wherein the one lengthwise cut is performed using a sterile surgical fine scissor and forceps.

4. The method of claim 1, further comprising placing the one or more precision-cut human bronchial tissue with the airway epithelium facing upwards onto one or more sterile surgical pathology sponges to establish the ex vivo model with an air-liquid interface condition in a 24-well culture plate.

5. The method of claim 4, wherein a culture medium is added to each well and the culture plate is incubated at 37° C. for subsequent viral infection experiments.

6. The method of claim 5, wherein the culture medium comprises F-12K nutrient mixture with L-glutamine and at least one antibiotic.

7. The method of claim 1, wherein the unwanted tissues comprise soft tissues and elastic blood vessels.

8. The method of claim 1, wherein the respiratory viral infections comprise influenza infection and coronavirus infection.

9. The method of claim 1, wherein the one or more respiratory viruses comprise influenza A viruses, influenza B viruses, coronaviruses, or any combinations of influenza viruses thereof.

10. The method of claim 1, wherein the virus infection is performed by subjecting the cilia-rich epithelium within the precision-cut human bronchial tissues to 10⁶ TCID₅₀/mL infectious dose of the one or more respiratory viruses and incubated at 37° C. and 5% CO₂ incubator.

11. The method of claim 1, the step of assessing infectivity, tropism, and pathogenesis of one or more respiratory viruses in the ex vivo model further comprises collecting tissue supernatants at 1 hour, 24 hours and 48 hours post-infection for measuring viral replication.

12. The method of claim 1, the step of assessing infectivity, tropism, and pathogenesis of one or more respiratory viruses in the ex vivo model further comprises fixing the precision-cut human bronchial tissues with 10% formalin at 24 hours post-infection or 48 hours post-infection in preparation for immunohistochemistry staining.

13. The method of claim 1, the step of providing a semi-quantitative and normalized approach comprising utilizing a post-staining or scoring analysis to supplement data from the ex vivo cultures of human bronchus.
